(19) United States
(12) Patent Application Publication
(10) Pub. No.: US 2012/0237610 A1
(43) Pub. Date: Sep. 20, 2012

(54) INJECTABLE COMPOSITIONS FOR INTRA-ARTICULAR USE COMBINING A VISCOSUPPLEMENTATION AGENT AND A FIBROBLAST GROWTH MEDIUM

(75) Inventors: Jean-Noël Thorel, Paris (FR); Hugues Gatto, Saint Paul De Vence (FR)

(73) Assignee: Jean-Noel THOREL, Paris (FR)

(21) Appl. No.: 13/512,948

(22) PCT Filed: Nov. 8, 2010
(86) PCT No.: PCT/FR2010/052397

§ 371 Date: May 31, 2012

(30) Foreign Application Priority Data
Dec. 18, 2009 (FR) 0959205

(51) Int. Cl. A61K 31/728; A61K 31/727; A61P 19/02; A61K 35/12; A61K 33/00; A61K 31/737; A61K 31/736

(52) U.S. Cl. 424/572; 514/54; 514/56; 514/57; 514/55; 424/600

(57) ABSTRACT

Provided is an injectable composition for intra-articular administration including at least one viscosupplementation agent selected from hyaluronic acid, chondroitin sulphate, keratin, keratin sulphate, heparin, cellulose and derivatives thereof, for example, chitosan, xanthans, galactomannan, alginates, and one or more salts thereof, and a fibroblast growth medium. The viscosupplementation agent and the fibroblast growth medium can be provided in a single composition for injection or as separate components for simultaneous, separate, or subsequent injection over time.

This invention concerns the development of solutions for intra-articular injection for the treatment of articular degeneration, in particular osteoarthritis.

It proposes combining a viscosupplementation agent, such as hyaluronic acid or one of its salts, with a fibroblast growth medium of defined composition, and possibly another polysaccharide, to advantage of natural origin.

### PRIOR ART

In some limb joints, the opposing bony extremities, protected by articular cartilage, are enclosed within a capsule lined by connective tissue, called the synovial membrane.

Synovial fluid is the viscous fluid which fills the articular cavity; it is composed of hyaluronic acid (HA), secreted by fibroblast cells of the synovial membrane (synoviocytes), and interstitial fluid filtered from the blood plasma. The functions of synovial fluid are to reduce the friction by lubricating the joint, absorb shocks, provide oxygen and nutrients to the chondrocytes of the articular cartilage, and eliminate carbon dioxide and metabolic waste from the latter, since the cartilage is not vascularised.

Osteoarthritis is a common degenerative articular condition with a multifactorial aetiology, involving loss of material from the articular cartilages. As the condition develops, a reduction is observed in the concentration and molecular weight of the HA present in the synovial fluid. This phenomenon is explained by a reduction in endogenous synthesis of hyaluronic acid and by the inflammation which generates free radicals, responsible for the oxidative degeneration.

These alterations cause a reduction in the viscoelastic properties of the HA and gradually lead to the loss of its essential function of protecting the joint. They can result in erosion of the cartilage, the presence of fragments of cartilage or bone within the articular cavity, pain and stiffness.

Viscosupplementation is a well-established therapeutic option consisting of injecting HA into the joint concerned to help lubricate it better, increase mobility and reduce pain. Depending on the severity of the osteoarthritis, series of 3 to 5 weekly injections, e.g. into the knee, are effective for 6 months to 1 year in the majority of patients. This therapeutic option is extremely useful, particularly for patients who do not tolerate, or no longer respond to conventional treatment such as anti-inflammatories and oral analgesics, but whose disease does not yet justify prosthetic treatment.

Improvement to viscosupplementation solutions has, up until now, concerned increasing the residence time of HA in the joint, with the objective of increasing its efficacy.

Processes of chemical modification of HA, cross-linking HA (WO 2007/070547), or combining HA with a polyol (WO 2009/024670) have been described for slowing down the in vivo mechanical or thermal degradation of the HA gel or its breakdown by radicals.

Nevertheless, the intra-articular persistence of HA viscosupplementation products, whether modified or not, the half-life of which is a few days, is still very much shorter than their period of therapeutic efficacy, which depends, indeed, on several cumulative roles:
a direct shock-absorber role for impacts on the cartilage;
properties capturing intra-articular debris, thus reducing its abrasive effect;
protection against inflammatory cells and the enzymes secreted by them; and
a possible direct action on pain receptors.

There is thus an obvious need to develop new therapeutic solutions for the treatment of articular degeneration, particularly of osteoarthritis.

### DESCRIPTION OF THE INVENTION

Given this situation, the Applicant has taken a completely new approach. It is intended that this invention should act at two distinct levels to re-establish good articular function, notably for treating articular degeneration, particularly osteoarthritis.

This invention therefore concerns a composition for injection combining a viscosupplementation agent and a fibroblast growth medium to revitalise the cellular components of the articular connective tissues, particularly synoviocytes and chondrocytes, and consequently ensure their cellular regeneration and stimulate their endogenous synthesis (of hyaluronic acid and GAGs, fundamental functional constituents of joints).

More precisely, the components likely to play the role of viscosupplementation agent in an intra-articular injectable composition according to the invention are chosen from the following list: hyaluronic acid, chondroitin sulphate, keratan, keratan sulphate, heparin, cellulose and its derivatives, chitosan, xanthans, galactomannan, the alginates and their respective salts.

In practice, the commonly used viscosupplementation agent in this intra-articular application is hyaluronic acid or one of its salts. For this reason, in a particular embodiment, the targeted composition only contains hyaluronic acid or one of its salts as the viscosupplementation agent, combined with the fibroblast growth medium.

An alternative embodiment would use hyaluronic acid or one of its salts as the main viscosupplementation agent in the planned composition, combined with at least one other polysaccharide, to advantage of natural origin to ensure its biocompatible, non-immunogenic character. This other polysaccharide is to advantage a polysulphated glycosaminoglycan—particularly chondroitin sulphate, keratan, keratan sulphate, or even heparin, cellulose and its derivatives, chitosan, the xanthans, galactomannan, the alginates and their respective salts. The composition contains in addition the fibroblast growth medium, and possibly other constituents.

In practice, it therefore involves combining a mechanical action of lubricating and protecting the joint with a trophic action of fibroblastic stimulation encouraging cell synthesis in the synovial membrane and articular cartilage. The first action is ensured by the viscosupplementation agent, to advantage hyaluronic acid—cross-linked or not, or one of its salts,—possibly combined with one or more other polysaccharides of natural origin. The second action is provided by the fibroblast growth medium as defined below.

As is known, the hyaluronic acid used in this invention may occur in different forms: as salts, derivatives such as esters or amides, and in a linear or chemically cross-linked form. All these forms can be envisaged for this invention. While cross-linking increases the lifespan of hyaluronic acid molecules within the organism, these modifications however affect its physical/chemical characteristics, biological properties and potential immunogenicity.

As indicated for hyaluronic acid, the polysaccharide or polysaccharides, to advantage of natural origin, may or may not be cross-linked, grafted or not grafted, using cross-linking and grafting techniques described in the prior art.

As a technical solution is required for the joint structures that is as neutral as possible, that is to say a biomimetic solution, non cross-linked hyaluronic acid, and its physiologically acceptable salts, are preferred for the first component as this molecule is a natural component of the synovial fluid. By physiologically acceptable salts of hyaluronic acid we mean particularly sodium and potassium salts, as well as mixtures of them.

The viscosupplementation agent, to advantage hyaluronic acid, is present in the composition preferably at a concentration of between 1 and 100 mg/ml, to advantage between 10 and 25 mg/ml.

The second essential component of the composition according to the invention is a fibroblast growth medium.

For this invention, a fibroblast growth medium is defined as a complete medium not only keeping fibroblasts alive but also stimulating their multiplication and synthesis within the cells (components of the extracellular matrix and synovial fluid).

Conducting a functional growth assay can determine whether a given medium is a fibroblast growth medium according to the invention. A suitable functional assay known to those working in the field is particularly colorimetric observation of the density of living cells using the reagent WST-1 and reading results at 450 nm (Berridge, M. V. et al. (1996): The Biochemical and Cellular Basis of Cell Proliferation Assays That Use Tetrazolium Salts. Biochemica 4, 15-19.)

As an example, a fibroblast growth medium is available commercially: this is the DMEM standard culture medium (Sigma) supplemented with 10% by weight of FCS (foetal calf serum) cell growth factor.

Generally speaking, such media contain extracts of animal or cellular origin which do indeed stimulate the growth of fibroblasts, but which have the disadvantage of not having a determined composition or of containing untraceable exogenous elements such as FCS, bovine pituitary extracts, the cell growth factors EGF (epidermal growth factor), FGF (fibroblast growth factor), insulin or cholera toxin, hydrocortisone, piperazine, etc.

To advantage, the fibroblast growth medium used in this invention does not contain cell growth factors or biological extracts of animal or cellular origin, in particular if these are not traced or traceable and/or are not of a defined composition.

The expression “not traced” or “not traceable” means that the source of the biological material in question and/or the treatment undergone by the latter cannot be established or checked.

In practice, the said medium to advantage contains no biological extract of animal or cellular origin, no cell compound or growth factor or hormone.

In a preferred embodiment, a fibroblast growth medium as compatible as possible with the natural environment of the joint, i.e. a medium containing biomimetic and/or biocompatible constituents (biological materials naturally present in the organism or neutral to it which do not induce allergic or inflammatory reactions), is introduced into the joint by intra-articular injection. To advantage, this medium includes components of the basic substance of connective tissue.

Such a medium will specifically provide fibroblasts with optimised nutrition in the form of vitamins, trace elements, amino acids, mineral salts, simple sugars (such as glucose, ribose, deoxyribose) and/or complex sugars (such as HA), and natural growth factors in the form of the constituents of nucleic acids (nitrogen containing bases and pentoses, needed to form nucleotides, and nucleosides). To advantage, it will also have a physiological pH between 6.5 and 7.9, preferably between 7.4 and 7.6 and an osmolarity between 280 and 450 mOsm, preferably between 300 and 350 mOsm.

It should be noted that HA can be both a component of the growth medium and the viscosupplementation agent. The difference is in the form of the HA (necessarily a physiological hyaluronate salt in the medium) and its quantity (much lower quantities in the medium).

To stimulate the growth of fibroblasts, such a medium can be enriched using a substance which is exogenous to the organism but of natural, traceable origin and well defined composition. A substance meeting this definition is for example a mixture of peptides extracted from milk, or MPC complex (Milk Peptide Complex), obtained by successive precipitation from milk then the separation of certain proteins subjected to enzyme hydrolysis.

This substance, in the form of a dehydrated powder, is added to the medium to advantage at between 0.5 to 5 mg/ml, to greater advantage at 4 to 5 mg/ml.

As an example, a complex medium meeting such a definition has been developed by the Applicant and combines about sixty components in precisely defined quantities as follows:

| INTERNATIONAL | |
|---|---|
| NOMENCLATURE OF COSMETIC | FINAL CONCENTRATION |
| INGREDIENTS NAME | Solution 1 X |
| (INCI) | (in mg/l) |
| WATER | q.s. 1 litre |
| SODIUM CHLORIDE | 5000 to 8000 |
| L-GLUTAMINE or | 100 to 3000 |
| L-ALANYL-GLUTAMINE | |
| SODIUM BICARBONATE | 0 to 2000 |
| D-GLUCOSE | 2000 to 5000 |
| L-ARGININE HCl | 300 to 500 |
| SODIUM ACETATE | 200 to 450 |
| DISODIUM PHOSPHATE Na₂HPO₄ | 100 to 1500 |
| L-LEUCINE | 50 to 200 |
| L-SERINE | 50 to 200 |
| MAGNESIUM CHLORIDE MgCl₂•6H₂O | 50 to 200 |
| POTASSIUM CHLORIDE | 50 to 200 |
| L-VALINE | 20 to 150 |
| SODIUM PYRUVATE | 10 to 75 |
| L-LYSINE HCl | 10 to 75 |
| L-HISTIDINE HCl•H₂O | 10 to 75 |
| L-CYSTEINE HCl•H₂O | 10 to 75 |
| ADENINE (HCl) | 5 to 50 |
| L-THREONINE | 5 to 50 |
| CALCIUM CHLORIDE CaCl₂•2H₂O | 0 to 22.5 |
| MYO-INOSITOL | 5 to 50 |
| L-GLUTAMIC ACID | 15 to 75 |
| L-ASPARAGINE H₂O | 15 to 75 |
| L-METHIONINE | 10 to 50 |
| L-TYROSINE 2Na₂2H₂O | 10 to 50 |
| L-PHENYLALANINE | 2 to 20 |
| L-TRYPTOPHAN | 2 to 20 |
| L-ALANINE | 5 to 30 |
| GLYCINE | 5 to 30 |
| L-ISOLEUCINE | 5 to 30 |
| L-ASPARTIC ACID | 10 to 50 |
| SODIUM SULPHATE | 1 to 10 |
| FERROUS SULPHATE FeSO₄•7H₂O | 1 to 10 |
| FOLIC ACID | 1 to 5 |
| THYMIDINE | 0.1 to 3 |
| CYANOCOBALAMINE | 0.1 to 3 |
| D-CALCIUM PANTOTHENATE | 1 to 5 |
| THIAMINE HCl | 1 to 5 |
| THIOCTIC ACID | 0.1 to 1 |
| ZINC SULPHATE ZnSO₄•7H₂O | 0.05 to 0.5 |
| SODIUM SILICATE Na₂SiO₃•4H₂O | 0.05 to 0.5 |
| PYRIDOXINE HCl | 0.5 to 3 |
| NIACINAMIDE (NICOTINAMIDE) | 0.5 to 3 |
| RIBOFLAVIN | 0.05 to 0.5 |
| d-BIOTIN | 0.01 to 0.05 |
| COPPER SULPHATE CuSO₄•5H₂O | 0 to 0.005 |
| AMMONIUM MOLYBDATE | 0 to 0.005 |
| (NH₄)₆Mo7O₂₄•4H₂O | |
| AMMONIUM VANADATE NH₄VO₃ | 0 to 0.001 |
| MANGANESE CHLORIDE MnCl₂•4H₂O | 0 to 0.0001 |
| SODIUM HYALURONATE | 100 to 1000 |
| L-PROLINE | 10 to 100 |
| HYDROXYPROLINE | 10 to 100 |
| ASCORBIC ACID | 0.1 to 10 |
| ADENOSINE | 0.01 to 1 |
| GUANINE | 0.01 to 1 |
| DEOXYRIBOSE | 0.01 to 1 |
| RIBOSE | 0.01 to 1 |
| CHOLINE CHLORIDE | 0 to 3 |
| MPC | 0 to 5000 |

As demonstrated below, such an enriched medium has a capacity in vitro to stimulate the growth of fibroblasts for several days. Moreover, it allows stimulated growth of the fibroblasts in the presence of serum. It is therefore a particularly suitable candidate for an intra-articular injection, in as far as part of the synovial fluid is a filtrate of blood plasma.

In addition, as demonstrated in this application, the incubation of fibroblasts in this medium increases the capacity of these cells to resist oxidative stress, i.e. it has anti-oxidant properties. Thus, in vitro, the enriched medium exerts an inhibitory effect on excess mitochondrial production of reactive oxygen species (superoxide ion) by fibroblasts exposed to a respiratory chain inhibitor (antimycin A). The expression kinetics of a fluorescent oxidation assay (DCFDA) is also significantly reduced for human fibroblasts pre-incubated in the growth medium and subjected to chemical oxidative stress, (AAPH), compared with control fibroblasts pre-incubated in standard DMEM. The anti-oxidant properties of the fibroblast growth medium also means that it has a role in protecting hyaluronic acid against oxidative degradation within the joint, which could increase the persistence of this compound in situ and prolong the therapeutic efficacy of the viscosupplementation.

Thus, the “fibroblast growth medium” according to the invention, which could also be called a “complete natural environment for the survival and growth of fibroblasts”, must have the following characteristics:
a/ a defined, traceable composition, containing only cell growth factors naturally present in the organism or neutral to it (amino acids, peptides, vitamins, trace elements, mineral salts, simple and complex sugars, nucleic acids), excluding any substance not of natural origin, of undefined composition or any drug substance;
b/ the ability, by itself, to enable the survival of fibroblasts in culture;
c/ and the ability to stimulate their growth and metabolism (and therefore production of material by the cells).

A composition according to the invention may in addition contain other ingredients or excipients, currently used in this application, particularly derivatives or purified fractions of HA. Nevertheless, according to a particular embodiment, the composition for injection consists only of the two components described above: firstly a viscosupplementation agent, to advantage hyaluronic acid possibly combined with one or more other polysaccharides of natural origin, and secondly a fibroblast culture medium.

As already stated, this composition is for intra-articular injection into a subject's joint cavity.

For this invention, the term “subject” designates a mammal, preferably a human, but may also designate an animal receiving veterinary treatment, particularly domestic animals or those used for recreational purposes (e.g. dogs, cat or horses).

In principle, all joints can be treated using a composition according to the invention. The knee is a joint particularly targeted in human subjects. In the dog, the hip is a joint frequently treated, while in the horse, to advantage it is the carpus, fetlock or hock.

According to a preferred embodiment, the composition, to advantage aqueous, is in the form of a gel, owing to application as an injection, the object of the invention. Remarkably, this restriction is perfectly compatible with the fibroblast growth media described above, which can be formulated as gels by incorporating hyaluronic acid, without adding exogenous excipients.

To even greater advantage, the composition is in the form of a monophasic hydrogel, i.e. a hydrogel as a single homogeneous phase. The viscosity of the composition obtained can be easily adjusted, particularly by adjusting the composition and the quantity of hyaluronic acid to obtain rheological properties similar to those of synovial fluid.

As an example, it has been shown that a composition according to the invention, with osmolarity between 300 and 350 mOsm, pH between 7.4 and 7.6 and a concentration of hyaluronic acid of molecular weight 1.3 to 1.8 MDa, of between 10 and 25 mg/ml was perfectly compatible with the application intended.

The composition for injection according to the invention may also form part of a kit including, in addition, syringes to contain the said composition. These may for example be single dose syringes of 2 to 20 ml. In such a kit, the 2 essential components of the composition can be presented as a mixture in the same syringe, or in 2 distinct syringes for extemporaneous mixing.

Given the injectable character and the intended treatment, such a composition is to advantage sterilised, cold sterilisation being used advantageously to avoid denaturing the components present. This may be performed by 0.22 μm membrane filtration for the fibroblast growth medium, and by separate sterilisation for the hyaluronic acid using a process known to those working in the field.

Another alternative consists of providing the composition for injection in the form of a powder (HA and fibroblast growth medium), in vials made of glass, polypropylene, polyethylene or any other material which can withstand sterilisation by ionising radiation or thermal flash sterilisation. In this embodiment, the monophasic hydrogel is reconstituted by adding sterile water to the vial, using a (sterile) syringe, before injecting the product into the joint. In this case, the product must be reconstituted between 2 to 72 hours before the injection.

Given their complementary mode of action, the two components of the composition according to the invention may be mixed and/or administered simultaneously, separately or spread over time.

A direct application for the composition according to the invention is treatment of articular degeneration, in particular osteoarthritis.

A composition according to the invention is therefore intended to be used as a medical device and/or medicinal product.

The invention will now be illustrated in a non-exhaustive manner by the following examples supported by the attached figures.

### LEGENDS OF FIGURES

FIG. 1 shows the comparative growth of human fibroblasts in culture in a fibroblast growth medium according to the invention and the DMEM standard medium (Sigma), without growth factor.

FIG. 2 shows the oxidation phenomena measured in a human fibroblast culture exposed to oxidative stress after incubation in different media.

### EXAMPLES OF EMBODIMENTS

### 1/ Use of a Fibroblast Growth Medium in a Composition for Injection

### a) Composition of the Medium

| INTERNATIONAL | |
|---|---|
| NOMENCLATURE OF COSMETIC | FINAL CONCENTRATION |
| INGREDIENTS NAME | Solution 1 X |
| (INCI) | (en mg/l) |
| WATER | q.s. 1 litre |
| SODIUM CHLORIDE | 5000 to 8000 |
| L-GLUTAMINE | 100 to 3000 |
| or L-ALANYL-GLUTAMINE | |
| SODIUM BICARBONATE | 0 to 2000 |
| D-GLUCOSE | 2000 to 5000 |
| L-ARGININE HCl | 300 to 500 |
| SODIUM ACETATE | 200 to 450 |
| DISODIUM PHOSPHATE Na₂HPO₄ | 100 to 1500 |
| L-LEUCINE | 50 to 200 |
| L-SERINE | 50 to 200 |
| MAGNESIUM CHLORIDE MgCl₂•6H₂O | 50 to 200 |
| POTASSIUM CHLORIDE | 50 to 200 |
| L-VALINE | 20 to 150 |
| SODIUM PYRUVATE | 10 to 75 |
| L-LYSINE HCl | 10 to 75 |
| L-HISTIDINE HCl•H₂O | 10 to 75 |
| L-CYSTEINE HCl•H₂O | 10 to 75 |
| ADENINE (HCl) | 5 to 50 |
| L-THREONINE | 5 to 50 |
| CALCIUM CHLORIDE CaCl₂•2H₂O | 0 to 22.5 |
| MYO-INOSITOL | 5 to 50 |
| L-GLUTAMIC ACID | 15 to 75 |
| L-ASPARAGINE H₂O | 15 to 75 |
| L-METHIONINE | 10 to 50 |
| L-TYROSINE 2Na₂2H₂O | 10 to 50 |
| L-PHENYLALANINE | 2 to 20 |
| L-TRYPTOPHAN | 2 to 20 |
| L-ALANINE | 5 to 30 |
| GLYCINE | 5 to 30 |
| L-ISOLEUCINE | 5 to 30 |
| L-ASPARTIC ACID | 10 to 50 |
| SODIUM SULPHATE | 1 to 10 |
| FERROUS SULPHATE FeSO₄•7H₂O | 1 to 10 |
| FOLIC ACID | 1 to 5 |
| THYMIDINE | 0.1 to 3 |
| CYANOCOBALAMINE | 0.1 to 3 |
| D-CALCIUM PANTOTHENATE | 1 to 5 |
| THIAMINE HCl | 1 to 5 |
| THIOCTIC ACID | 0.1 to 1 |
| ZINC SULPHATE ZnSO₄•7H₂O | 0.05 to 0.5 |
| SODIUM SILICATE Na₂SiO₃•4H₂O | 0.05 to 0.5 |
| PYRIDOXINE HCl | 0.5 to 3 |
| NIACINAMIDE (NICOTINAMIDE) | 0.5 to 3 |
| RIBOFLAVIN | 0.05 to 0.5 |
| d-BIOTIN | 0.01 to 0.05 |
| COPPER SULPHATE CuSO₄•5H₂O | 0 to 0.005 |
| AMMONIUM MOLYBDATE | 0 to 0.005 |
| (NH₄)₆Mo7O₂₄•4H₂O | |
| AMMONIUM VANADATE NH₄VO₃ | 0 to 0.001 |
| MANGANESE CHLORIDE MnCl₂•4H₂O | 0 to 0.0001 |
| SODIUM HYALURONATE | 100 to 1000 |
| L-PROLINE | 10 to 100 |
| HYDROXYPROLINE | 10 to 100 |
| ASCORBIC ACID | 0.1 to 10 |
| ADENOSINE | 0.01 to 1 |
| GUANINE | 0.01 to 1 |
| DEOXYRIBOSE | 0.01 to 1 |
| RIBOSE | 0.01 to 1 |
| CHOLINE CHLORIDE | 0 to 3 |
| MPC | 0 to 5000 |

### b) Human Fibroblast Culture

Protocol
Human fibroblasts were seeded at a low density in 96-well plates in a DMEM standard culture medium, supplemented with FCS (foetal calf serum) cell growth factor.
After 24 h, they were cultured in the pure medium according to the invention or in the DMEM standard medium without growth factor.
The media were not renewed during the experiment.
The density of living cells was determined at T0 then after 2, 4, 7 and 9 days, using a colorimetric method (WST-1 reagent).

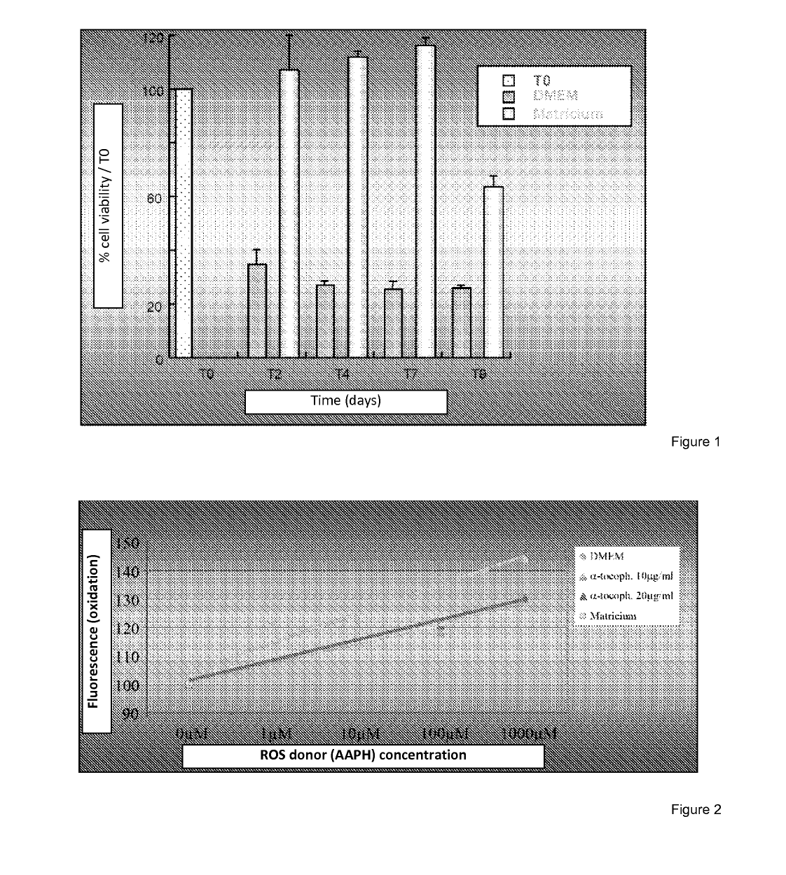

Results
The culture medium according to the invention alone maintained the growth of the fibroblasts over a period of 9 days. From the 7^{th} day slowing of cell growth was observed which can be explained by the fact that the medium was not renewed (FIG. 1).
In the DMEM medium without FCS, a reduction in cell viability was seen after 2 days and cell growth was absent throughout of the study (FIG. 1).

In conclusion, it appears that the fibroblast growth medium used according to the invention allows survival and stimulates the growth of normal human fibroblasts in the absence of exogenous growth factors.

### c) Increase in the Resistance of Fibroblasts to Oxidative Stress

Protocol
Normal human fibroblasts in the growth phase were seeded into 96-well plates, in DMEM augmented with 10% foetal calf serum.
48 h later they were put into DMEM (negative control), into the medium according to the invention, or α-tocopherol (positive anti-oxidant control) for 2 h.
They were rinsed and incubated for 25 min with a massive ROS donor (oxidative stress with AAPH) and a detector (DCFDA) which becomes fluorescent when oxidised.
The rate of appearance of fluorescence (proportional to the production of cellular ROS) was measured every 3 minutes.

Results
Significant inhibition of the fluorescence (oxidation) for the cells pre-incubated in the medium according to the invention (Matricium) compared with the DMEM negative control (FIG. 2). The medium according to the invention increases the capacity of the cells to withstand oxidative stress.
Indirect inhibitory effect on oxidative stress (improvement in the cells' redox homeostasis) comparable in intensity with that of an anti-oxidant with a classic direct effect (α-tocopherol which captures free radicals).

In conclusion, it is evident that the medium exerts an inhibitory effect on excess mitochondrial production of reactive oxygen species (superoxide ion) by fibroblasts exposed to a respiratory chain inhibitor (antimycin A). The expression kinetics of a fluorescent oxidation assay (DCFDA) is also significantly reduced for human fibroblasts pre-incubated in the growth medium and subjected to chemical oxidative stress, (AAPH), compared with control fibroblasts pre-incubated in standard DMEM.

### 2/ Preparation of an Injectable Gel for Intra-Articular Injection

Fibroblast growth medium.
HA is added at a concentration of between 1 and 100 mg/ml and preferably at a concentration of between 10 and 25 mg/ml.
Formulation of a Gel: the hyaluronic acid (HA) is dissolved in the fibroblast culture medium. The HA concentration determines the viscosity of the final preparation. As an example, the HA used is sodium hyaluronate with a molecular weight between 1.3 and 1.8 MDa. The gel for injection according to the invention does not contain any additive, all the components of the formula acting both as excipients and active ingredients.
Sterilisation: by 0.22 μm membrane filtration for the fibroblast growth medium, and by separate sterilisation using a process known to those working in the field for the HA. Another alternative consists of providing the composition for injection as a powder (HA and fibroblast growth medium), in vials which can withstand sterilisation using ionising radiation or a thermal flash technique. The monophasic hydrogel is reconstituted by adding sterile water to the vial, before the product is injected into the joint.
Injection Protocol: depending on the joint to be treated and the severity of the osteoarthritis, one injection per week is recommended for 3 to 5 weeks. The length of action depends on the severity of the articular lesions and the subject's age.

1.-13. (canceled)

14. A composition for intra-articular injection, comprising:
at least one viscosupplementation agent selected from the group consisting of hyaluronic acid, chondroitin sulphate, keratan, keratan sulphate, heparin, cellulose and its derivatives, chitosan, a xanthan, galactomannan, an alginate and a salt thereof; and
a fibroblast growth medium free of any cell growth factor or any biological extract of animal or cellular origin which has not been traced and/or is of undefined composition.

15. The composition according to claim 14, wherein the viscosupplementation agent is hyaluronic acid or a salt thereof.

16. The composition according to claim 15, further comprising at least one other polysaccharide.

17. The composition according to claim 16, wherein the at least one other polysaccharide is of natural origin.

18. The composition according to claim 17, wherein the at least one other polysaccharide of natural origin is selected from the group consisting of chondroitin sulphate, keratan, keratan sulphate, heparin, cellulose and its derivatives, chitosan, a xanthan, galactomannan, an alginate and a salt thereof.

19. The composition according to claim 14, in the form of a gel.

20. The composition according to claim 19, wherein the gel is a sterile aqueous gel.

21. The composition according to claim 14, wherein the viscosupplementation agent is present in the composition at a concentration of between 1 and 100 mg/ml.

22. The composition according to claim 21, wherein the viscosupplementation agent is hyaluronic acid.

23. The composition according to claim 14, wherein the viscosupplementation agent is present in the composition at a concentration of between 10 and 25 mg/ml.

24. The composition according to claim 23, wherein the viscosupplementation agent is hyaluronic acid.

25. The composition according to claim 14, wherein the fibroblast growth medium comprises components of connective tissue.

26. The composition according to claim 25, wherein the fibroblast growth medium further comprises a mixture of milk peptides (MPC).

27. The composition according to claim 14, wherein the fibroblast growth medium comprises:
constituents of nucleic acids;
amino acids;
simple and complex sugars;
vitamins; and
an inorganic fraction containing trace elements and mineral salts.

28. A kit comprising syringes comprising the composition according to claim 14.

29. A medical device comprising the composition according to claim 14.

30. The composition according to claim 14, for the treatment of articular degeneration.

31. The composition according to claim 30, wherein the articular degeneration is osteoarthritis.

32. A method for treating articular degeneration in a subject, comprising administering to the subject, a therapeutically effective amount of the composition according to claim 14.

33. The method of claim 32, wherein the articular degeneration is osteoarthritis.

34. The method of claim 32, wherein administering comprises injecting the composition into the subjects joint cavity.

35. The composition according to claim 14 for treating articular degeneration, wherein the at least one viscosupplementation agent and the fibroblast growth medium are separate components that can each be injected simultaneously or subsequently over time.

36. The kit according to claim 28, for the treatment of articular degeneration.

37. The kit according to claim 35, wherein the articular degeneration is osteoarthritis.

38. The medical device according to claim 29, for the treatment of articular degeneration.

39. The medical device according to claim 38, wherein the articular degeneration is osteoarthritis.
